(19) United States
(12) Patent Application Publication
(10) Pub. No.: US 2003/0216628 A1
(43) Pub. Date: Nov. 20, 2003

(54) Methods and systems for assessing glycemic control using predetermined pattern label analysis of blood glucose readings

(76) Inventors: Jonathan David Bortz, St. Louis, MO (US); Eric Stephen Armbrecht, St. Louis, MO (US)

Correspondence Address: Michael T. Marrah, Sonnenschein Nath & Rosenthal, Wacker Drive Station, Sears Tower, P.O. Box #061080, Chicago, IL, 60606-1080, US

(21) Appl. No.: 10/059,084

(22) Filed: Jan. 28, 2002

(51) Int. Cl. G01N 31/00; G06F 19/00; A61B 5/00; G06F 17/60

(52) U.S. Cl. 600/365; 702/22; 600/319; 705/2

(57) ABSTRACT

Methods and systems for analyzing blood glucose readings comprising the steps of obtaining a plurality of blood glucose readings taken within a predetermined time category and time period, performing first calculations on said readings based on a predetermined normal range of glycemia in a first analysis and selecting and applying a pattern label having predetermined criteria to the plurality of blood glucose readings by comparing the results of the first calculations to the pattern label criteria. The invention may also include the steps of performing second calculations on said readings based on predetermined thresholds for severe hyperglycemia and severe hypoglycemia and selecting and appending a severity suffix having predetermined severity criteria to said pattern label by comparing the results of the second calculations to the severity criteria as well as performing third calculations on said readings based on a predetermined normal range of glycemia and selecting and appending a minor comment having minor comment criteria to said pattern label by comparing the results of the third calculations to the comment criteria.

### FIELD OF THE INVENTION

The present invention is in the field of chemical arts, specifically, the field of blood glucose level analysis.

### BACKGROUND OF THE INVENTION

Proper analysis of blood glucose levels is crucial to providing optimal care to diabetic patients. However, proper analysis of blood glucose levels for even a single patient requires the analysis of mountains of individual readings from various time categories taken over various spans to determine the clinical significance of the individual readings and any information shown by groups of readings. Such analysis is time-consuming and tedious for medical professionals to perform on any significant scale.

Moreover, the lack of generally accepted analytical terms for analyzing the raw blood glucose reading data increases the complexity of the analysis and burdens the exchange of analysis data between medical professionals. This makes it more difficult for medical professionals to correlate a given series of readings with the proper course of medical intervention. Of course, it also makes educating patients about their own condition and treatment options difficult.

### SUMMARY OF THE INVENTION

The invention is a method and computer system for analyzing blood glucose readings comprising the steps of obtaining a plurality of blood glucose readings taken within a predetermined time category and time period, performing calculations on said readings and selecting and applying a pattern label having predetermined criteria to the plurality of blood glucose readings by comparing the results of the calculations to the pattern label criteria.

Such embodiment may also include the steps of performing second calculations on said readings based on predetermined thresholds for severe hyperglycemia and severe hypoglycemia and comparing the results of said second calculations to predetermined severity criteria and selecting and appending a severity suffix to said pattern label or determining that no severity suffix is necessary. The invention may also include performing third calculations on said readings based on a predetermined normal range of glycemia and comparing the results of the third calculations to predetermined minor comment criteria to select and append a minor comment to said pattern label based.

In light of the foregoing and the following description of the invention, it is one object of the present invention to provide a method for analyzing a plurality of blood glucose readings by assigning pattern labels—clinically significant but previously undefined terms which identify clinically actionable distributions of blood glucose measurements—to categories of the readings and outputting a glucose control assessment based thereon.

It is another object of the invention to provide automated, computer implemented systems for the implementation of the inventive method.

Other objects and advantages of the present invention are identified in the drawings, specification and claims herein or will otherwise be apparent to those skilled in the art. While the invention has been disclosed herein in various embodiments and examples, it is subject to various modifications and alternative forms. Nothing in this specification is intended to or may be interpreted to limit the scope of the invention as determined by the broadest possible interpretation of the appended claims.

### BRIEF DESCRIPTION OF THE DRAWINGS

FIG. 1 is a flow chart of one embodiment of the present inventive method and system;

FIG. 2 shows the default pattern labels and pattern label criteria;

FIG. 3 shows the default severity suffixes and severity suffix criteria;

FIG. 4 shows the default minor comments and minor comment criteria;

FIG. 5 is an example of one possible output of the inventive system showing a sample assessment of glycemic control for a hypothetical patient with pattern labels for various time categories;

FIG. 6 is another exemplar output of the inventive system showing a sample assessment and report of glycemic control for a hypothetical patient with pattern labels for various time categories;

FIG. 7 is an output report of the system showing sample raw blood glucose reading data and patient provided comments; and

FIG. 8 shows calculation results used in one embodiment of the invention.

### DETAILED DESCRIPTION OF THE PREFERRED EMBODIMENTS

The methods and systems of the present invention classify infinite possibilities of blood glucose readings into a finite number of clinically meaningful statements about a patient's glycemic control. As such, the invention assists medical professionals to efficiently and consistently assess a patient's glycemic control and administer proper clinical intervention as necessary.

A preferred embodiment of the invention comprises a computer system implementing the inventive method for automatically analyzing blood glucose readings in a given time category and time period comprising the steps of obtaining a plurality of blood glucose readings taken within a predetermined time category and time period, performing calculations on said readings based on a predetermined normal range of glycemia and selecting a pattern label having predetermined criteria by comparing said criteria to said calculation results to assess a human patient's glycemic control.

The present invention also provides for severity checks and commentary, called severity suffixes, based on analysis of the plurality of readings in the time category. In this way, both the overall pattern of the readings and discrete severe readings are analyzed and reported on by the method and system. In addition, the invention may provide a minor comment, for example, where severity suffixes are not necessary, to further explain the readings. Thus, the full pattern label of the present invention may comprise the base pattern label, a severity suffix (if selected), and/or a minor comment.

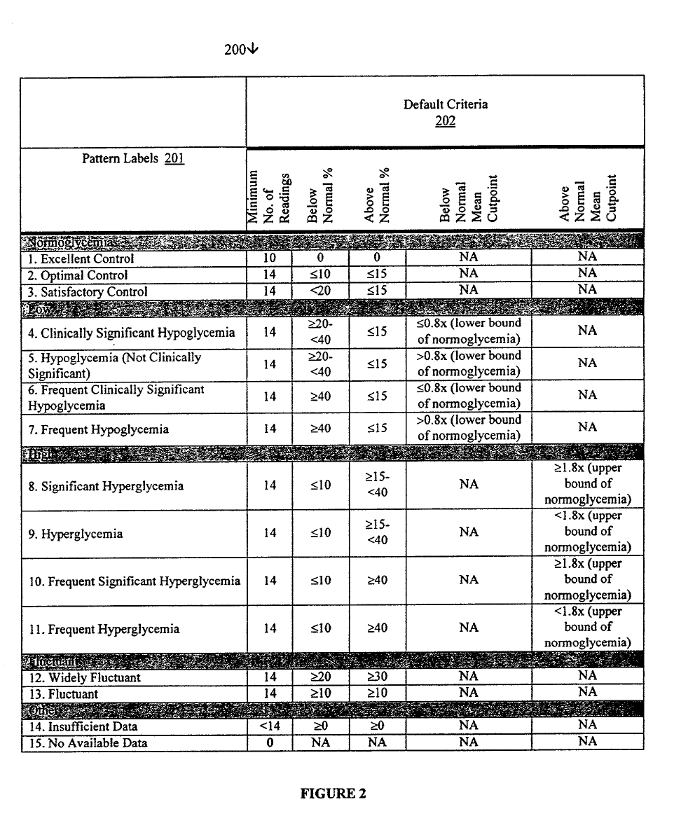

The blood glucose patterns of the present invention comprise clinically significant terms which identify clinically actionable distributions of blood glucose measurements but which do not presently have a generally accepted definitions within the art of glucose reading analysis. The default and preferred set of standardized pattern labels and label criteria of the invention are shown in FIG. 2.

By analyzing the glucose reading data via patterns rather than statistical calculations of the data (e.g., standard deviations, means, skewness, etc.), the method achieves clinical significance in a manner that can be understood by the lay healthcare provider. Patterns, as opposed to statistical calculations, represent a more natural analysis of glucose readings when blood glucose readings are used for treatment decisions by health care providers.

The analysis procedure is conducted by a computer system that obtains daily blood glucose readings and other data necessary for treatment decisions through multiple channels. Blood glucose reading data may be submitted to a database by manual data entry, over the Internet, by telephone or on a paper form. In addition, data may be obtained directly from the diagnostic device that analyzed the blood to yield the reading. Depending on the preferences of the user, an output report may be displayed on a computer screen, or communicated via fax, email or postal mail. Such a report may display the raw data, graphical representations of the data and a glycemic control report as determined by the invention.

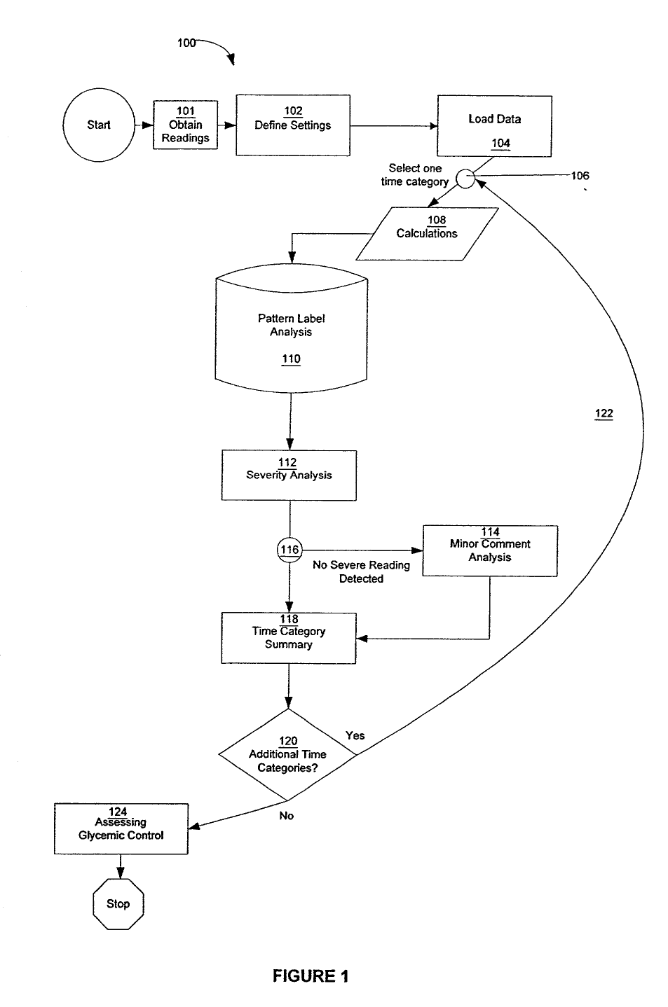

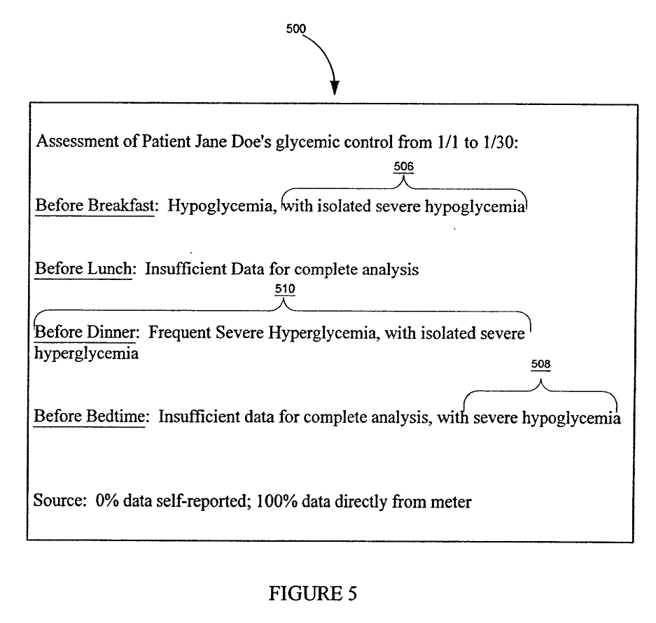

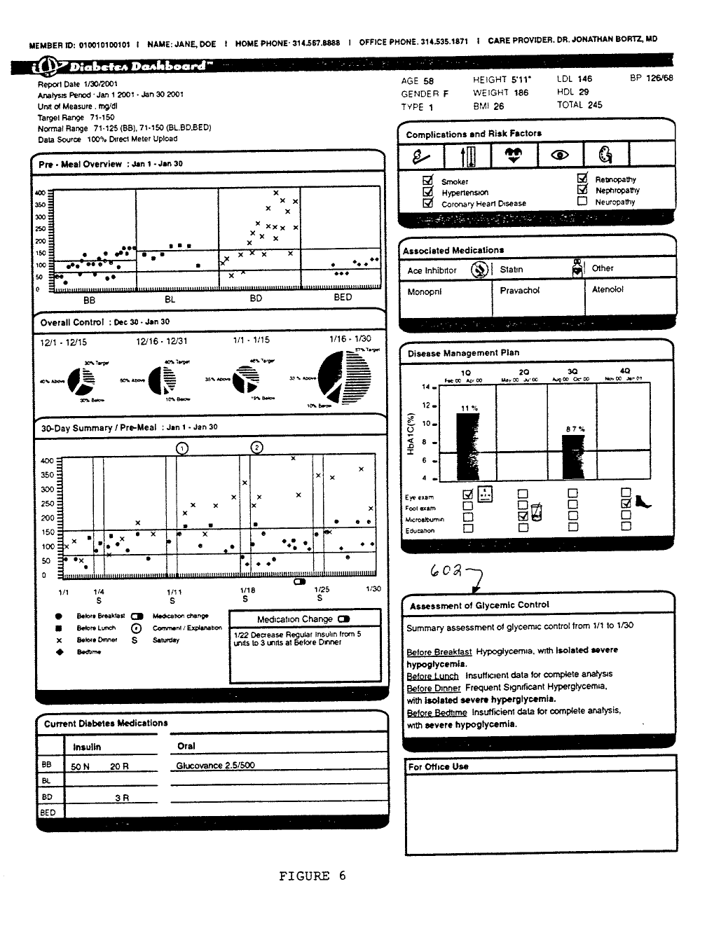

FIG. 1 shows the general process 100 of one embodiment of the present invention culminating in assessing glycemic control 124. A glycemic control report is constructed by analyzing the data for each time category separately. Examples of such a report are shown in FIGS. 5 and 6 as references 500 and 602.

The first step in the method involves obtaining a plurality of blood glucose readings 101. Either before or after the blood glucose readings are made, a user selects 102 desired settings preferably via computer software driven menus. Such settings may include selecting the analysis time period, the time category under investigation 106, the data weighting desired and the normoglycemia range.

The analysis time period is the date range over which blood glucose readings to be analyzed are taken. A Begin Date and End Date may be set to cover any time period. The default time period may be 30 days and may be adjusted by the user as necessary.

Time categories are recognized as intra-day periods in which blood glucose readings are taken. The present invention accommodates at least the following standardized and preferred time categories:

| | |
|---|---|
| Before Breakfast | After Breakfast |
| Before Lunch | After Lunch |
| Before Dinner | After Dinner |
| Before Bedtime | Nighttime |

Although such standardized categories are specified in the system by default, they may be modified or amended as necessary by the user.

The system or a data collection instrument (e.g., web-enabled software application, paper-based logbook, glucometer etc.) manages or at least facilitates the system's allocation of glucose reading data to one of the time categories. When multiple time categories are to be analyzed, each is analyzed independently and the resulting pattern labels combined in the assessing glycemic control step 124 for the time period.

Data weighting is a method by which some readings, for example, more recent blood glucose readings, are given greater influence in the analysis. This may be accomplished by multiplying each reading over a specified period of time (e.g., the most recent 10 days) by a positive integer, thereby changing the influence of the glucose readings during that period in the overall analysis. Fox example, if a dataset contain 30 blood glucose readings (one on each day at the same time category of the specified analysis period) and 10 glucose readings from the most recent 10 days of the analysis period are given a weight of 2, then each of the glucose readings during that specified period would count twice as much as the others in the data set. The weighting convention is made available to account for the relative importance of recent glucose readings over older glucose readings.

In defining settings 102, the user may also define the blood glucose range which represents the normal range of glycemia or normoglycemia. The default and preferred standardized normal range is 71-150 mg/dl for all time categories except Before Breakfast. The default Before Breakfast normal range is 71-125 mg/dl. The normal range may be modified by the medical professional for each patient, each time period and at each time category, but once set, is the same for all analysis done within each time category.

Once the settings 102 are established and the blood glucose readings are obtained, the blood glucose readings are loaded 104 into the computer system. This may be accomplished in many ways known in the art, including via the Internet, modem transfer, scanner/digitizer, upload from a glucometer or even direct data entry. Then, if not already established in the settings 102 step, the user may select the appropriate time category 106 at issue in order to filter the relevant readings for analysis.

As the invention uses customizable pattern labels having customizable criteria, alternative sets of labels and corresponding criteria may be selected prior to analysis 110, for example, at the defined settings 102 step. Shown in FIG. 2 is a representation of the default pattern label set 200 used for the inventive analysis. The default set of labels and criteria, shown in FIG. 2, are preferred and provide the highest level of standardization of analysis. However, the labels 201 and their attendant criteria 202 may be customized by the medical professional within the limit of the need to create mutually exclusive criteria and labels. Customization of the pattern label set 200 may occur at the patient-level or at a system level that would be applicable to all patients. The customized labels 201 and/or criteria 202 may be maintained by the system in sets akin to the default set 200 which are selectable by the user.

For example, “Optimal Control” could be defined differently for an obese, Type 2 diabetic versus a 23-year old woman with gestational diabetes. Therefore, a system administrator may modify the “Optimal Control” label criteria to make the former patient's control requirements more stringent, thereby creating a custom label set for that patient or type of patient. Thus, the present invention affords healthcare providers the flexibility to define their own terms for analysis to ensure proper care.

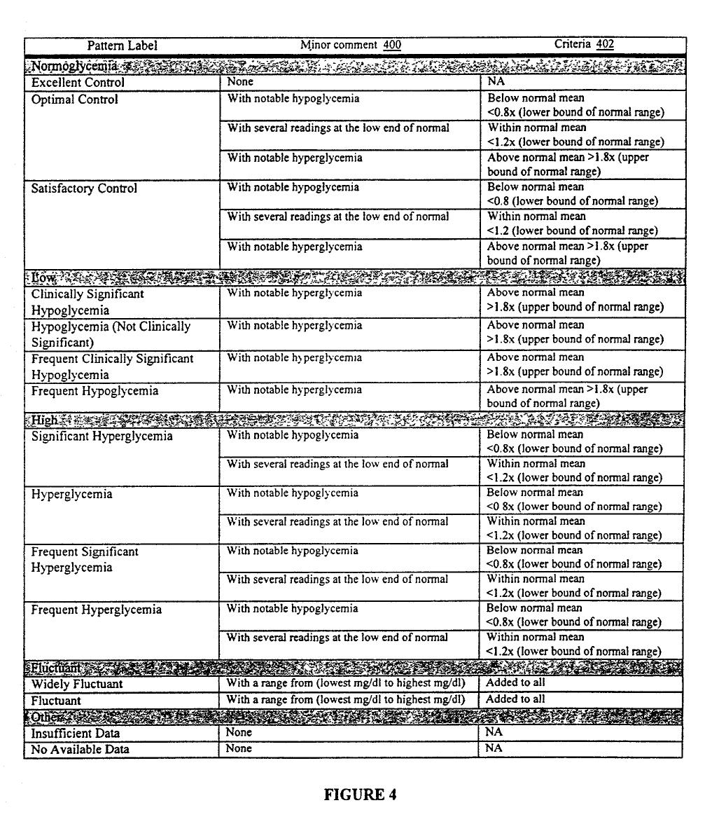

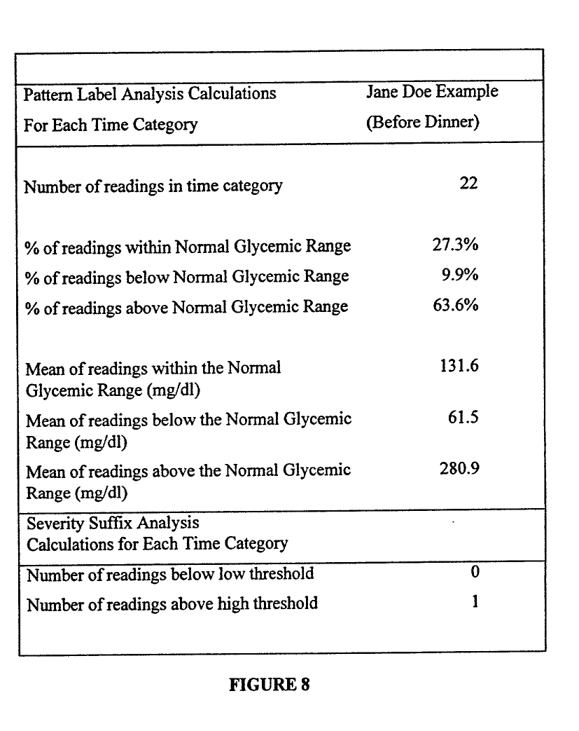

In the next step, various calculations 108 are performed on the readings to provide for the selection of the pattern label. A preferred embodiment of the calculations is illustrated, with exemplar results, in FIG. 8. The calculations may include counting the number of readings in the time category for the time period; computing the percentages of readings above, below and within the normal range of glycemia; and computing the means of readings above, below and within the normoglycemia range. For the severity suffix analysis 112, they may include counting the number of blood glucose readings above and below predetermined high and low threshold values of glycemia. High and low thresholds may be based on the numerical distance from the upper and lower bounds of the normal range of glycemia. Other thresholds used in the calculations, for example, those for the minor comments as shown in FIG. 4, may also be based on the upper and lower bounds of the normal range of glycemia.

To ensure relevancy of the calculation, a minimum number of readings must occur in the defined time period in order for the assessing 124 to take place. The preferred number of readings necessary to support application of the various pattern labels 201 for a reasonable analysis of the readings are shown in FIG. 2 under the heading “Minimum No. of Readings.”

The percentage of readings that fall within the default preferred standardized normal range of glycemia (e.g., 71-150 mg/dl) may be calculated by dividing the number of readings that occur within normal range by the total number of readings in the time category and multiplying that number by 100. The percentage of readings that fall below normal range may also be calculated for the time category by dividing the number of readings that occur below normal by the total number of readings multiplied by 100. The percentage of readings that fall above the normal range may be calculated for the time category by dividing the number of readings that occur above normal by the total number of readings multiplied by 100.

The mean of readings that fall within the normal range may be calculated for the time category by summing the values of all the normal range readings and dividing by the total number of normal range readings. The mean of readings that fall below (e.g., <71 mg/dl) the normal range may be calculated for the time category by summing the values of all the below normal range readings and dividing by the total number of below normal range readings. The mean of readings that fall above (e.g., >150 mg/dl) the defined normal range may be calculated for the time category by summing the values of all the above normal range readings and dividing by the total number of above range readings. In this way, the lay user comprehension of the pattern of blood glucose readings may be enhanced as the analysis is expressed as percentages rather than complicated statistics such as standard deviations and other measures of variance, correlation or central tendency.

To select a pattern label, the calculation results may be compared to the pattern label criteria 202 for the selected label set, in a step-wise manner. A patient's glucose readings for each time category may be filtered through the criteria using the following logic:

“if the calculations on a patient's collected glucose readings match the predetermined criteria for pattern label x, then report pattern label x.”

For example, the calculation results are compared to the criteria for the Excellent Control label. If all the criteria fit the results, the label is selected and attached to the dataset. If not, the criteria are compared to the Optimal Control label and so on through the pattern label set 200 until the calculation results match a single pattern label criteria.

While a step-wise selection system is used in the preferred embodiment, it is understood that other known methods of data-analysis may also be suitable. For example, rather than comparing the results to the criteria for an individual label, the system may compare the results to an entire class of criteria (i.e. Minimum No. of Readings, Below Normal Percentage, etc.) across the various pattern labels at a time. Thus, the system would filter out the single appropriate pattern as each class of criteria filters out the inappropriate labels.

Severity Suffix

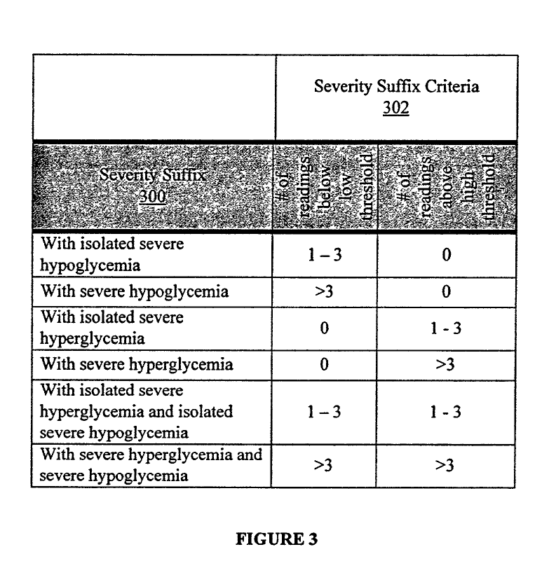

The system and method may also include second calculations for a Severity Analysis 112 for extreme blood glucose readings. The default set 301 of Severity Suffixes 300 and their criteria 302 are shown in FIG. 3. The preferred second calculations for the Severity Analysis 112 are shown in FIG. 8. Such analysis may comprise conducting second calculations and comparing the results of those second calculations to the Severe Suffix criteria 302 to identify and select a severity suffix (generally 300, with a specific examples shown as 506 and 508 in FIG. 5) to append to the pattern label. The number of blood glucose readings that occur below the severe hypoglycemia threshold may also be counted for the time category. The default severe hypoglycemia threshold is 40 mg/dl. Also, the number of blood glucose readings that occur above the severe hyperglycemia threshold may be counted for the time category. The default severe hyperglycemia threshold is 400 mg/dl.

As with the pattern label analysis 110, the severity analysis 112 may comprise a step-wise comparison of the severity suffix criteria 302 and a pattern label severity suffix 300 is added to the pattern label in the presence of severely hyperglycemic or hypoglycemic readings. In the absence of such readings, i.e., where the severity suffix criteria 302 are not met 116, no suffix may be added and a minor comment analysis 114 may be conducted. Minor comment The invention may also conduct a minor comment analysis 114 based on the blood glucose readings to paint a more complete picture of the patient's glycemic control when no severity suffix is appropriate 116. Table 4 identifies the various default minor comments 400 and the criteria 402 used by the system to append them to the pattern label using the step-wise procedure previously discussed. In general, the minor comments 400 provide greater detail to the analysis provided by the pattern label itself. This additional modifying clause to the pattern label gives the healthcare provider additional information that might otherwise be overlooked.

Third calculations, those for the minor comment analysis, may comprise calculating the mean of readings below a normal range of glycemia (“below normal mean” in FIG. 4), the mean of readings within a normal range of glycemia (“within normal mean”), and the mean of readings above a normal range of glycemia (“above normal mean”). Other third calculations may include computing the thresholds used in the minor comment criteria. For example, the criteria for the “with notable hypoglycemia” minor comment for the pattern label “Optimal Control,” utilizes a threshold of 0.8 multiplied by the lower bound of the predetermined normal range of glycemia. Thus, a data set having an Optimal Control pattern label with a “below normal mean” of less than that threshold, would result in the selection of the “with notable hypoglycemia” minor comment. Of course, many, if not all, of the minor comment calculations may be done when the calculations for the pattern label and/or severity suffix are made.

As with the pattern label itself, the severity suffix and minor comment, as well as their respective criteria, are customizable within the limit of having each label and its respective criteria mutually exclusive of the others.

Once the pattern label 110, severity suffix 112 and minor comment analyses 114, if any, are complete, the complete pattern label 510 for the time category is assembled in the time category summary step 118.

The method described above is repeated 120, 122 for all other time categories until all desired time categories have been analyzed whereupon the analysis proceeds to assessing glycemic control 124, which may comprise compiling the pattern labels for all the time categories analyzed and creating at least one glycemic control report 500 for the period analyzed as shown in FIG. 5.

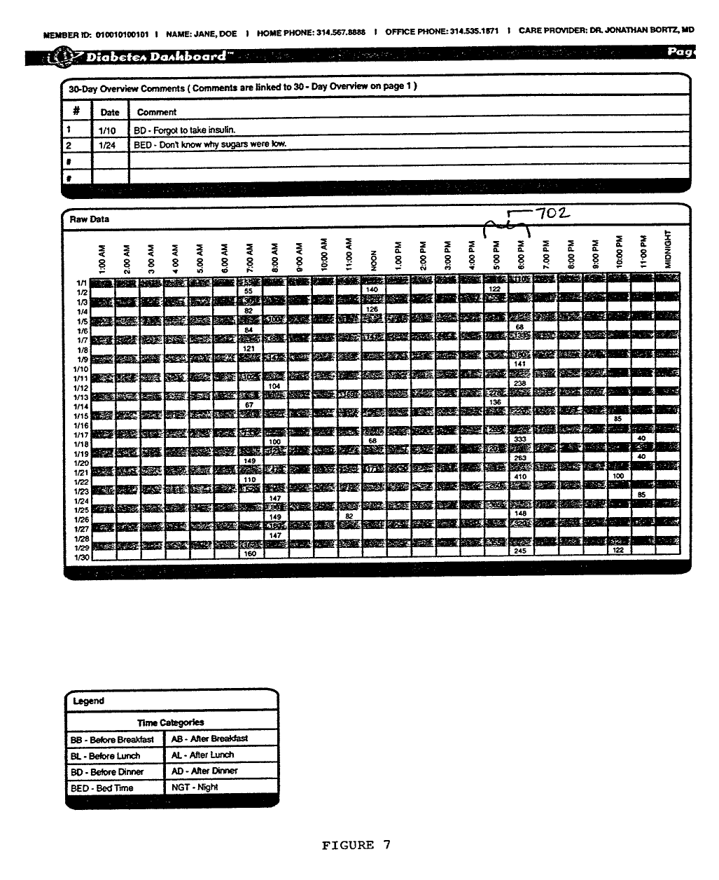

In this way, raw blood glucose readings, such as those shown in report format in FIG. 7, are converted from raw data to an easy-to-read, clinically meaningful glycemic control report 500, to assist the medical professionals in diabetes treatment and the patients in educating themselves as to their conditions. Examples of such reports are shown as reference numerals 500 and 602 in FIGS. 5 and 6, respectively. FIG. 6 also illustrates a glycemic control report 602 as part of a larger data report presenting other glycemic control data. FIG. 7 shows the specific raw data 702 used to provide the report 602.

### EXAMPLE

To further explain a preferred embodiment of the present invention, the following example is presented with the calculation results discussed herein charted in FIG. 8. Jane Doe, a hypothetical patient, is a middle-aged Type I diabetic who tests her blood four times during the day: before breakfast, before lunch, before dinner, and before bedtime. Of the 30 days between January 1 and January 30, she tested and records her blood glucose levels 22 times before dinner (data shown as columns 702 in FIG. 7). She uploaded her data into the inventive system via her glucometer, her home computer, and an Internet connection. Her physician has elected to use the default, preferred pattern label sets, normoglycemia range, severity suffix set and minor comment label and criteria. For purposes of example, the analysis of data from the before dinner time category is discussed herein.

Jane Doe's before dinner blood glucose readings are:

[image]

The system calculations are made as follows: six of the 22 readings are between 71-150 mg/dl. Thus, 27.3% of Before Dinner readings from January 1 to January 30 are normal.

In the Jane Doe example, 2 of the 22 readings in FIG. 1 are below 71 mg/dl. Thus, 9.9% of Before Dinner readings from January 1 to January 30 are below normal.

In the Jane Doe example, 14 of the 22 readings in FIG. 1 are above 150 mg/dl. Thus, 63.6% of Before Dinner readings from January 1 to January 30 are above normal.

In the Jane Doe example, the within-normal range mean equals 131.6 mg/dl calculated as follows:

Σ(110, 122, 133, 136, 141, 148)=790. The sum of 790 divided by the number of normal readings (6) is equal to the mean 131.6 mg/dl.

In the Jane Doe example, the below-normal range mean equals 61.5 mg/dl calculated as follows:

Σ(55, 68)=123. The sum of 123 divided by the number of readings above the normal (2) is equal to the mean 61.5 mg/dl.

In the Jane Doe example, the above-normal range mean equals 280.9 mg/dl calculated as follows:

Σ(180, 201, 220, 238, 245, 260, 263, 265, 278, 320, 333, 355, 365, 410)=3933. The sum of 3933 divided by the number of readings above the normal (14) is equal to the mean 280.9 mg/dl.

There are no readings below 40 mg/dl in the Jane Doe example.

There is one reading above 400 mg/dl in the Jane Doe example.

Once the calculations are made, the resultant values are compared to the pattern label set criteria in a stepwise manner. The first pattern label criteria of the default set 200 that completely “fits” the patient's data is selected as the best pattern label. The results of the calculations on Jane Doe's raw data that are necessary for the pattern label selection process are shown below and in FIG. 8:

Count of Readings during Analysis Period=22

% of readings below normal=9.9%

% of readings above normal=63.6%

Mean of Below Normal Readings=61.5 mg/dl

Mean of Above Normal Readings=280.9 mg/dl

The best fitting pattern label for Jane Doe for the Before Dinner time category is “Frequent Significant Hyperglycemia,” determined as follows. First, the calculation results are compared to the criteria for “Excellent Control.” The first requirement of a minimum of 10 readings is met. However, Jane Doe does not meet the Below Normal percentage criteria because the Below Normal percentage of 9.9% is greater than the required 0%. Therefore, this pattern label is excluded and the process proceeds to the next—Optimal Control.

Jane Doe meets the minimum readings requirement of 14 for “Optimal Control”, but does not meet the Above Normal percentage criteria because 63.6% (Jane Doe) is greater than 15%(criteria). Therefore, “Optimal Control” is not selected. The process continues until a complete match is found.

As the analysis procedure continues, all of the low blood glucose patterns are rejected.

Based on the percentage of readings above normal, frequent hyperglycemia is apparent. The choice between “Frequent Significant Hyperglycemia” and “Frequent Hyperglycemia” is made using the Above Normal mean. Jane Doe's Above Normal Mean (280.9 mg/dl) is greater than the cut-point (270 mg/dl) thereby distinguishing her readings as elevated enough to be called “significant.” Therefore, “Frequent Significant Hyperglycemia” is selected as the best pattern label to describe Jane Doe's before dinner glycemic control.

After the pattern label is selected, the data is filtered through the severe reading analysis.

As shown, one of Jane Doe's readings is above 400 mg/dl. The severe reading check would therefore append the suffix “with isolated severe hyperglycemia” to the pattern label 510 for the Before Dinner category.

We claim:

1. A method for the assessment of glycemic control of a human patient using blood glucose readings comprising the steps of:
(1) obtaining a plurality of blood glucose readings taken within a predetermined time category and time period;
(2) performing first calculations on said readings based on a predetermined normal range of glycemia; and
(3) selecting a pattern label having predetermined criteria by comparing the results of said first calculations to the predetermined pattern label criteria to assess the glycemic control of a human patient.

2. The method of claim 1 further comprising the steps of:
performing second calculations on said readings based on predetermined thresholds for severe hyperglycemia and severe hypoglycemia; and
if the results of said second calculations meet a severity criteria, selecting and appending a severity suffix having predetermined severity criteria to said pattern label by comparing the results of said second calculations to the severity criteria.

3. The method of claim 2 further comprising the steps of:
performing third calculations on said readings based on a predetermined normal range of glycemia; and
if the results of said third calculations meet a minor comment criteria, selecting and appending a minor comment having predetermined minor comment criteria to said pattern label by comparing the results of said third calculations to said comment criteria.

4. The method of claim 1 further comprising the step of outputting said pattern label for said time category as at least part of a glycemic control report.

5. The method of claim 3 further comprising the step of outputting said pattern label for said time category, including any appended severity suffixes and minor comments, as part or all of a glycemic control report.

6. The method of claim 1 further comprising the step of repeating method steps (1), (2) and (3) for time categories or time periods other than the predetermined time category and time period.

7. The method of claim 6 further comprising the step of outputting a pattern label for each time category as part or all of a glycemic control report.

8. The method of claim 5 further comprising the step of repeating the method steps for time categories or time periods other than the predetermined time category and time period.

9. The method for analyzing blood glucose readings of claim 1 wherein said pattern label is chosen from a pattern label set comprising at least the following labels and criteria:

| | Default Criteria | | | | |
|---|---|---|---|---|---|
| | | | | Below | Above |
| | Minimum | | | Normal | Normal |
| Pattern | No. of | Below | Above | Mean | Mean |
| Labels | Readings | Normal % | Normal % | Cutpoint | Cutpoint |
| Normoglycemia | | | | | |
| 1. Excellent Control | 10 | 0 | 0 | NA | NA |
| 2. Optimal Control | 14 | ≦10 | ≦15 | NA | NA |
| 3. Satisfactory Control | 14 | <20 | ≦15 | NA | NA |
| Low | | | | | |
| 4. Clinically Significant | 14 | ≧20- | ≦15 | ≦0.8 × (lower bound | NA |
| Hypoglycemia | | <40 | | of normoglycemia) | |
| 5. Hypoglycemia (Not | 14 | ≧20- | ≦15 | >0.8 × (lower bound | NA |
| Clinically Significant) | | <40 | | of normoglycemia) | |
| 6. Frequent Clinically | 14 | ≧40 | ≦15 | ≦0.8 × (lower bound | NA |
| Significant Hypoglycemia | | | | of normoglycemia) | |
| 7. Frequent Hypoglycemia | 14 | ≧40 | ≦15 | >0.8 × (lower bound | NA |
| | | | | of normoglycemia) | |
| High | | | | | |
| 8. Significant | 14 | ≦10 | ≧15- | NA | ≧1.8 × |
| Hyperglycemia | | | <40 | | (upper |
| | | | | | bound of |
| | | | | | normogly |
| | | | | | cemia) |
| 9. Hyperglycemia | 14 | ≦10 | ≧15- | NA | <1.8 × |
| | | | <40 | | (upper |
| | | | | | bound of |
| | | | | | normogly |
| | | | | | cemia) |
| 10. Frequent Significant | 14 | ≦10 | ≧40 | NA | ≧1.8 × |
| Hyperglycemia | | | | | (upper |
| | | | | | bound of |
| | | | | | normogly |
| | | | | | cemia) |
| 11. Frequent | 14 | ≦10 | ≧40 | NA | <1.8 × |
| Hyperglycemia | | | | | (upper |
| | | | | | normogly |
| | | | | | cemia) |
| Fluctuant | | | | | |
| 12. Widely Fluctuant | 14 | ≧20 | ≧30 | NA | NA |
| 13. Fluctuant | 14 | ≧10 | ≧10 | NA | NA |
| Other | | | | | |
| 14. Insufficient Data | <14 | ≧0 | ≧0 | NA | NA |
| 15. No Available Data | 0 | NA | NA | NA | NA |

10. The method of claim 2 wherein said severity suffix is selected from the suffix set comprising the following suffixes and criteria:
Severity Suffix Criteria

| | # of | # of |
|---|---|---|
| | readings | readings |
| | below | above |
| | low | high |
| Severity Suffix | threshold | threshold |
| With isolated severe | 1-3 | 0 |
| hypoglycemia | | |
| With severe hypoglycemia | >3 | 0 |
| With isolated severe | 0 | 1-3 |
| hyperglycemia | | |
| With severe hyperglycemia | 0 | >3 |
| With isolated severe | 1-3 | 1-3 |
| hyperglycemia and isolated | | |
| severe hypoglycemia | | |
| With severe hyperglycemia and | >3 | >3 |
| severe hypoglycemia | | |

11. The method of claim 3 wherein said minor comment is selected from the comment set comprising the following comments and criteria:

| Pattern Label | Minor comment | Criteria |
|---|---|---|
| Normoglycemia | | |
| Excellent Control | None | NA |
| Optimal Control | With notable hypoglycemia | Below normal mean |
| | | <0.8 × (lower bound |
| | | of normal range) |
| | With several readings at the | Within normal mean |
| | low end of normal | <1.2 × (lower bound |
| | | of normal range) |
| | With notable hyperglycemia | Above normal mean |
| | | >1.8 × (upper bound |
| | | of normal range) |
| Satisfactory | With notable hypoglycemia | Below normal mean |
| Control | | <0.8 (lower bound |
| | | of normal range) |
| | With several readings at the | Within normal mean |
| | low end of normal | <1.2 (lower bound |
| | | of normal range) |
| | With notable hyperglycemia | Above normal mean |
| | | >1.8 × (upper bound |
| | | of normal range) |
| Low | | |
| Clinically | With notable hyperglycemia | Above normal mean |
| Significant | | >1.8 × (upper bound |
| Hypoglycemia | | of normal range) |
| Hypoglycemia | With notable hyperglycemia | Above normal mean |
| (Not Clinically | | >1.8 × (upper bound |
| Significant) | | of normal range) |
| Frequent | With notable hyperglycemia | Above normal mean |
| Clinically | | >1.8 × (upper bound |
| Significant | | of normal range) |
| Hypoglycemia | | |
| Frequent | With notable hyperglycemia | Above normal mean |
| Hypoglycemia | | >1.8 × (upper bound |
| | | of normal range) |
| High | | |
| Significant | With notable hypoglycemia | Below normal mean |
| Hyperglycemia | | <0.8 × (lower bound |
| | | of normal range) |
| | With several readings at | Within normal mean |
| | the low end of normal | <1.2 × (lower bound |
| | | of normal range) |
| Hyperglycemia | With notable hypoglycemia | Below normal mean |
| | | <0.8 × (lower bound |
| | | of normal range) |
| | With several readings at the | Within normal mean |
| | low end of normal | <1.2 × (lower bound |
| | | of normal range) |
| Frequent | With notable hypoglycemia | Below normal mean |
| Significant | | <0.8 × (lower bound |
| Hyperglycemia | | of normal range) |
| | With several readings at the | Within normal mean |
| | low end of normal | <1.2 × (lower bound |
| | | of normal range) |
| Frequent | With notable hypoglycemia | Below normal mean |
| Hyperglycemia | | <0.8 × (lower bound |
| | | of normal range) |
| | With several readings at the | Within normal mean |
| | low end of normal | <1.2 × (lower bound |
| | | of normal range) |
| Fluctuant | | |
| Widely | With a range from (lowest mg/ | Added to all |
| Fluctuant | dl to highest mg/dl) | |
| Fluctuant | With a range from (lowest mg/ | Added to all |
| | dl to highest mg/dl) | |
| Other | | |
| Insufficient | None | NA |
| Data | | |
| No Available | None | NA |
| Data | | |

12. The method of claim 1 wherein said first calculations comprise computing:
The number of readings in the time category within the time period;
The percentage of readings within the normal range of glycemia;
The percentage of readings above the normal range of glycemia;
The percentage of readings below the normal range of glycemia;
The mean of readings within the normal range of glycemia;
The mean of readings above the normal range of glycemia; and
The mean of readings below the normal range of glycemia.

13. The method of claim 2 wherein said second calculations comprise counting the number of readings above a predetermined high glycemia threshold and the number below a low glycemia threshold.

14. The method of claim 3 wherein said third calculations comprise calculating the mean of readings above normal glycemia, the mean of readings below normal glycemia, and the mean of readings within a normal range of glycemia.

15. The method of analyzing blood glucose readings of claim 1 wherein the step of performing calculations on said readings further comprises the steps of:
Counting the number of readings in the time category within the time period;
Calculating the quantity of readings above, below and within predetermined normal range of glycemia;
Calculating the mean of readings above, the mean below and the mean within a predetermined normal range of glycemia; and
Calculating the quantity of readings outside predetermined severe glycemia thresholds.

16. A glycemic control report comprising a pattern label for a predetermined time category within a predetermined time period.

17. A glycemic control report comprising a pattern label and severity suffix for a predetermined time category within a predetermined time period.

18. A glycemic control report comprising a pattern label and a minor comment for a predetermined time category within a predetermined time period.

19. A computer system for the assessment of glycemic control using the method of claim 1.

20. A computer system for the assessment of glycemic control using the method of claim 2.

21. A computer system for the assessment of glycemic control using the method of claim 3.

22. A computer system for the assessment of glycemic control using the method of claim 4.

23. A computer system for the assessment of glycemic control using the method of claim 5.

24. A computer system for the assessment of glycemic control using the method of claim 6.

25. A computer system for the assessment of glycemic control using the method of claim 7.

26. A computer system for the assessment of glycemic control using the method of claim 8.

27. A computer for creating a glycemic control report of claim 16.

28. A computer for creating a glycemic control report of claim 17.

29. A computer for creating a glycemic control report of claim 18.

30. A computer-readable medium containing instructions to cause a computer system to perform the method of claim 1.

31. A computer-readable medium containing instructions to cause a computer system to perform the method of claim 2.

32. A computer-readable medium containing instructions to cause a computer system to perform the method of claim 3.

33. A computer-readable medium containing instructions to cause a computer system to perform the method of claim 4.

34. A computer-readable medium containing instructions to cause a computer system to perform the method of claim 5.

35. A computer-readable medium containing instructions to cause a computer system to perform the method of claim 6.

36. A computer-readable medium containing instructions to cause a computer system to perform the method of claim 7.

37. A computer-readable medium containing instructions to cause a computer system to perform the method of claim 8.
